(19) United States
(12) Patent Application Publication
(10) Pub. No.: US 2024/0315531 A1
(43) Pub. Date: Sep. 26, 2024

(54) ENDOSCOPIC IMAGE OBSERVATION SUPPORT DEVICE AND ENDOSCOPE SYSTEM

(71) Applicant: FUJIFILM Corporation, Tokyo (JP)

(72) Inventor: Kentaro OSHIRO, Kanagawa (JP)

(73) Assignee: FUJIFILM Corporation, Tokyo (JP)

(21) Appl. No.: 18/735,153

(22) Filed: Jun. 5, 2024

Related U.S. Application Data
(63) Continuation of application No. PCT/JP2022/039849, filed on Oct. 26, 2022.

(30) Foreign Application Priority Data
Dec. 13, 2021 (JP) 2021-201741

(51) Int. Cl. A61B 1/00

(52) U.S. Cl. CPC A61B 1/000094; A61B 1/0005

(57) ABSTRACT

Provided are an endoscopic image observation support device and an endoscope system that can provide a user interface with high visibility even if having a plurality of support functions. The endoscopic image observation support device supports observation of an image captured by an endoscope and includes a processor. The processor is configured to cause a display device to display the image. In addition, the processor is configured to set degrees of priority of a plurality of pieces of support information to be displayed on the display device, and cause the display device to display the plurality of pieces of support information based on the degrees of priority.

### CROSS-REFERENCE TO RELATED APPLICATIONS

The present application is a Continuation of PCT International Application No. PCT/JP2022/039849 filed on Oct. 26, 2022 claiming priority under 35 U.S.C § 119(a) to Japanese Patent Application No. 2021-201741 filed on Dec. 13, 2021. Each of the above applications is hereby expressly incorporated by reference, in its entirety, into the present application.

### BACKGROUND OF THE INVENTION

### 1. Field of the Invention

The present invention relates to an endoscopic image observation support device and an endoscope system, and in particular, to an endoscopic image observation support device and an endoscope system that support observation of an image captured by an endoscope.

### 2. Description of the Related Art

In recent years, in the field of medical endoscopes, observation support techniques using artificial intelligence (AI) have been being developed. For example, JP2019-180966A, JP2020-175051A, WO2020/105699A describe techniques for supporting detection, discrimination, and the like of a lesion by using AI. By providing various support functions to a user, it is possible to reduce the observation load.

### SUMMARY OF THE INVENTION

However, if the number of support functions to be provided increases, the amount of information to be displayed on a screen increases, and there is a problem in that the visibility of a user interface decreases.

An embodiment according to the technology of the present disclosure provides an endoscopic image observation support device and an endoscope system that can provide a user interface with high visibility even if having a plurality of support functions.

(1) An endoscopic image observation support device that supports observation of an image captured by an endoscope, the endoscopic image observation support device including: a processor configured to: cause a display device to display the image; set degrees of priority of a plurality of pieces of support information to be displayed on the display device; and cause the display device to display the plurality of pieces of support information based on the degrees of priority.

(2) The endoscopic image observation support device according to (1), in which the plurality of pieces of support information include at least one of information indicating a position of a lesion part, information indicating a discrimination result, or information indicating an observation progress state.

(3) The endoscopic image observation support device according to (2), in which the information indicating an observation progress state is displayed using a progress bar.

(4) The endoscopic image observation support device according to (2), in which the information indicating an observation progress state is displayed using a schema diagram of an observation target organ.

(5) The endoscopic image observation support device according to any one of (1) to (4), in which the processor is configured to cause the display device to display the plurality of pieces of support information at positions in accordance with the degrees of priority.

(6) The endoscopic image observation support device according to any one of (1) to (5), in which the processor is configured to cause the display device to display the plurality of pieces of support information in sizes in accordance with the degrees of priority.

(7) The endoscopic image observation support device according to any one of (1) to (6), in which the processor is configured to cause the display device to display the plurality of pieces of support information at luminances in accordance with the degrees of priority.

(8) The endoscopic image observation support device according to any one of (1) to (7), in which the processor is configured to emphasize display of one of the plurality of pieces of support information with a degree of priority higher than a threshold value.

(9) The endoscopic image observation support device according to (8), in which the processor is configured to blink display of the one of the plurality of pieces of support information with the degree of priority higher than the threshold value.

(10) The endoscopic image observation support device according to any one of (1) to (9), in which the processor is configured to hide display of one of the plurality of pieces of support information with a degree of priority lower than a threshold value.

(11) The endoscopic image observation support device according to any one of (1) to (10), in which the processor is configured to change the degrees of priority in accordance with an operation state of the endoscope.

(12) The endoscopic image observation support device according to (11), in which the processor is configured to determine the operation state of the endoscope, based on the image.

(13) The endoscopic image observation support device according to (11) or (12), in which the operation state of the endoscope is whether an observed site and/or a lesion part is being observed.

(14) The endoscopic image observation support device according to any one of (11) to (13), in which the operation state of the endoscope is whether a region in which a lesion part is detectable is being observed.

(15) The endoscopic image observation support device according to any one of (11) to (14), in which the operation state of the endoscope is whether there is an unobserved site.

(16) The endoscopic image observation support device according to any one of (11) to (15), in which the operation state of the endoscope is whether treatment is being performed.

(17) The endoscopic image observation support device according to any one of (1) to (16), in which the processor is configured to change the degrees of priority when display content of a specific piece of support information is updated.

(18) An endoscope system including: an endoscope; a display device; and the endoscopic image observation support device according to any one of (1) to (17).

According to the present invention, it is possible to provide a user interface with high visibility even if having a plurality of support functions.

### BRIEF DESCRIPTION OF THE DRAWINGS

FIG. 1 is a block diagram illustrating an example of a system configuration of an endoscope system;

FIG. 2 is a block diagram of main functions of a processor device;

FIG. 3 is a block diagram illustrating an example of a hardware configuration of an endoscopic image observation support device;

FIG. 4 is a block diagram of main functions of the endoscopic image observation support device;

FIG. 5 is a block diagram of main functions of an image recognition processing unit;

FIG. 6 is a diagram illustrating an example of observation progress state determination results;

FIG. 7 is a diagram illustrating an example of a table;

FIG. 8 is a diagram illustrating an example of screen display;

FIG. 9 is a diagram illustrating an example of display of second support information;

FIG. 10 is a diagram illustrating an example of display of the second support information;

FIG. 11 is a diagram illustrating an example of display of third support information;

FIG. 12 is a flowchart illustrating a procedure of a support information displaying process;

FIG. 13 is a diagram illustrating an example of a case where luminance is changed in accordance with degrees of priority for display;

FIG. 14 is a diagram illustrating an example of a case where first support information is displayed in an emphasized manner;

FIG. 15 is a diagram illustrating another example of the third support information;

FIG. 16 is a diagram illustrating an example of display of an observation state display map on a screen;

FIG. 17 is a diagram illustrating an example of display of support information in accordance with degrees of priority;

FIG. 18 is a diagram illustrating another example of display of support information in accordance with degrees of priority;

FIG. 19 is a diagram illustrating an example of a case where the third support information is displayed in an emphasized manner; and

FIG. 20 is a block diagram of main functions of the endoscopic image observation support device.

### DESCRIPTION OF THE PREFERRED EMBODIMENTS

Hereinafter, preferred embodiments of the present invention will be described in detail with reference to the accompanying drawings.

### First Embodiment

Here, a case will be described as an example in which the present invention is applied to an endoscope system that performs an endoscopic examination of an upper digestive organ, in particular, a stomach.

### Configuration of Endoscope System

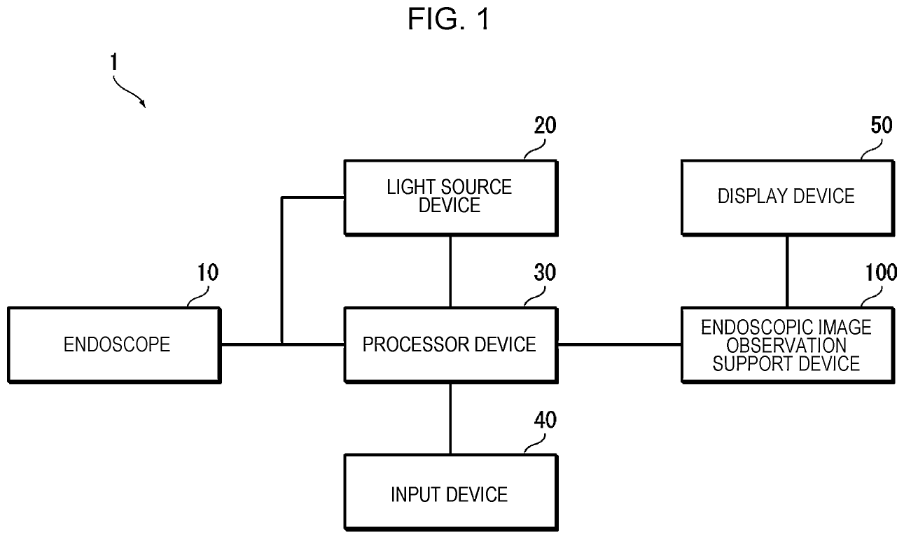

FIG. 1 is a block diagram illustrating an example of a system configuration of the endoscope system.

As illustrated in FIG. 1, an endoscope system 1 according to this embodiment includes an endoscope 10, a light source device 20, a processor device 30, an input device 40, a display device 50, an endoscopic image observation support device 100, and the like. The endoscope 10 is connected to the light source device 20 and the processor device 30. The light source device 20, the input device 40, and the endoscopic image observation support device 100 are connected to the processor device 30. The display device 50 is connected to the endoscopic image observation support device 100.

The endoscope system 1 according to this embodiment is configured as a system by which observation using special light (special-light observation) is possible in addition to observation using normal white light (white-light observation). The special-light observation includes narrow-band light observation. The narrow-band light observation includes blue laser imaging observation (BLI observation), narrow band imaging observation (NBI observation), linked color imaging observation (LCI observation), and the like. Note that the special-light observation itself is a known technique, and thus, detailed description thereof is omitted.

### Endoscope

The endoscope 10 according to this embodiment is an electronic endoscope (flexible endoscope), in particular, an electronic endoscope for the upper digestive organ. The electronic endoscope includes an operating unit, an insertion unit, a connection unit, and the like, and images a subject with an imaging element incorporated in a distal end of the insertion unit. The operating unit includes a forceps port in addition to operating members such as an angle knob, an air/water supply button, a suction button, a mode switching button and a release button. The mode switching button is a button for switching an observation mode. For example, switching is performed among a mode for white-light observation, a mode for LCI observation, and a mode for BLI observation. The release button is a button for issuing an instruction for capturing a still image. Note that the endoscope itself is known, and thus, detailed description thereof is omitted. The endoscope 10 is connected to the light source device 20 and the processor device 30 via the connection unit.

### Light Source Device

The light source device 20 generates illumination light to be supplied to the endoscope 10. As described above, the endoscope system 1 according to this embodiment is configured as a system by which the special-light observation is possible in addition to the normal white-light observation. Thus, the light source device 20 has a function of generating light (e.g., narrow-band light) compatible with the special-light observation in addition to the normal white light. Note that, as described above, the special-light observation itself is a known technique, and thus, description of generation of the illumination light is omitted. The light source type is switched, for example, by the mode switching button provided in the operating unit of the endoscope 10.

### Processor Device

The processor device 30 integrally controls the operation of the entire endoscope system 1. The processor device 30 includes, as its hardware configuration, a processor, a main memory, an auxiliary storage, an input/output interface, an operation panel, and the like. The processor is constituted by, for example, a central processing unit (CPU). The main memory is constituted by, for example, a random access memory (RAM). The auxiliary storage is constituted by, for example, a hard disk drive (HDD), a solid state drive (SSD), or the like. The operation panel is provided with various operation buttons.

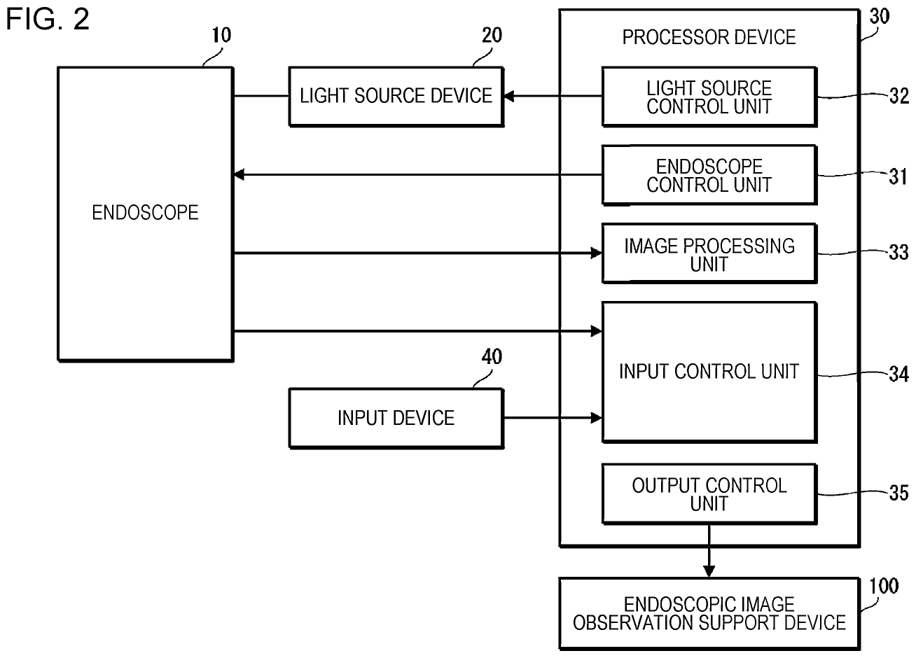

FIG. 2 is a block diagram of main functions of the processor device.

As illustrated in FIG. 2, the processor device 30 has functions of an endoscope control unit 31, a light source control unit 32, an image processing unit 33, an input control unit 34, an output control unit 35, and the like. Each function is implemented by the processor executing a predetermined program. The auxiliary storage stores various programs to be executed by the processor, various kinds of data necessary for control, and the like.

The endoscope control unit 31 controls the endoscope 10. The control of the endoscope 10 includes driving control of the imaging element, control of air/water supply, control of suction, and the like.

The light source control unit 32 controls the light source device 20. The control of the light source device 20 includes light emission control of the light source, switching control of the light source type, and the like.

The image processing unit 33 performs processing of generating a captured image by performing various kinds of signal processing on a signal output from the imaging element of the endoscope 10.

The input control unit 34 performs processing of receiving an input of an operation from the input device 40 and the operating unit of the endoscope 10 and an input of various kinds of information.

The output control unit 35 controls output of information to the endoscopic image observation support device 100. The information output to the endoscopic image observation support device 100 includes, in addition to an image captured by the endoscope, information input through the input device 40, various kinds of operation information, and the like. The various kinds of operation information include, in addition to operation information by the input device 40, operation information by the operating unit of the endoscope 10, operation information of the operation panel provided in the processor device 30, and the like.

### Input Device

The input device 40 is constituted by a keyboard, a foot switch, or the like. Note that instead of the keyboard or the like, or in addition to the keyboard or the like, the input device 40 can also be constituted by a touch panel, an audio input device, a line-of-sight input device, or the like.

### Display Device

The display device 50 is constituted by a flat panel display (FPD) such as a liquid crystal display (LCD) or an organic light emitting diode (OLED) display. An image captured by the endoscope is displayed on the display device 50. Note that instead of the FPD or in addition to the FPD, the display device 50 can also be constituted by a head mounted display (HMD), a projector, or the like.

### Endoscopic Image Observation Support Device

The endoscopic image observation support device 100 causes the display device 50 to display an image captured by the endoscope and provides a user with observation support functions. In this embodiment, as the observation support functions, a function of supporting detection of a lesion part, a function of supporting discrimination, and a function of reporting an observation progress state are provided.

The function of supporting detection of a lesion part is provided as a function of automatically detecting a lesion part from an image captured by the endoscope 10 and reporting the position of the lesion part on a screen of the display device 50.

The function of supporting discrimination is provided as a function of discriminating a detected lesion part and reporting the result on the screen of the display device 50.

The function of reporting an observation progress state is provided as a function of reporting, if a site to be observed is determined, the observation progress state on the screen of the display device 50.

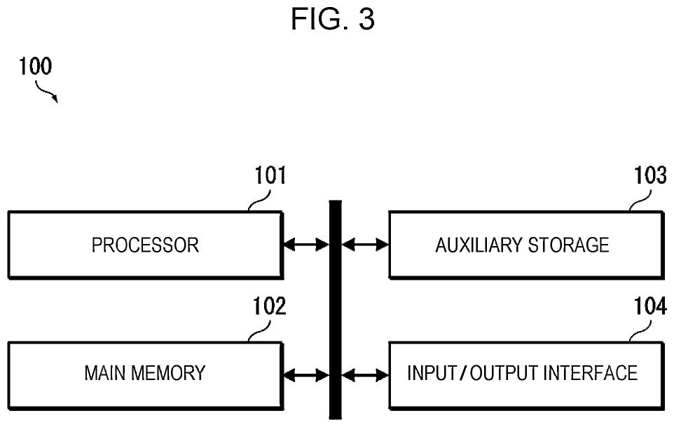

FIG. 3 is a block diagram illustrating an example of a hardware configuration of the endoscopic image observation support device.

The endoscopic image observation support device 100 is constituted by a so-called computer and includes, as its hardware configuration, a processor 101, a main memory 102, an auxiliary storage 103, an input/output interface 104, and the like. The endoscopic image observation support device 100 is connected to the processor device 30 and the display device 50 via the input/output interface 104. The processor is constituted by, for example, a CPU. The main memory 102 is constituted by, for example, a RAM. The auxiliary storage 103 is constituted by, for example, an HDD, an SSD, or the like. The auxiliary storage 103 stores programs to be executed by the processor 101 and various kinds of data necessary for control or the like. In addition, information such as an image captured by the endoscope or a recognition processing result is recorded in the auxiliary storage 103.

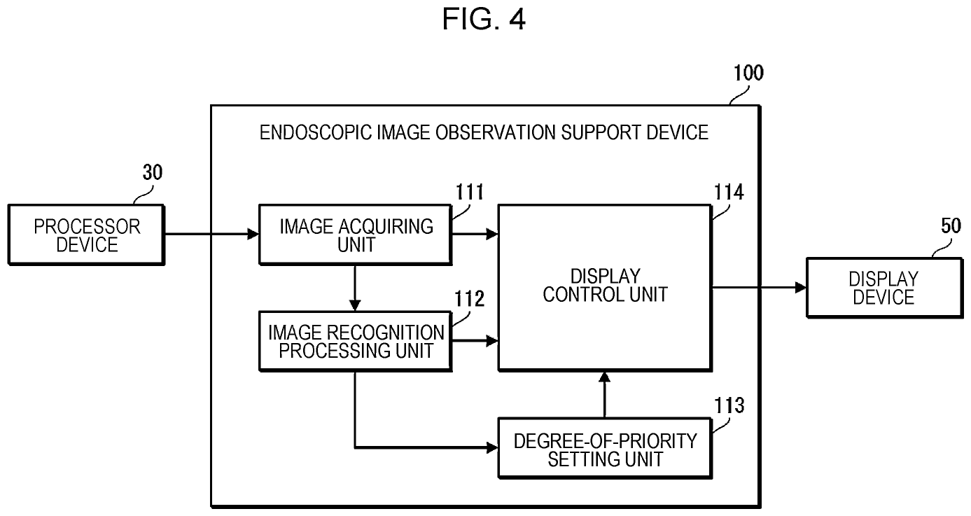

FIG. 4 is a block diagram of main functions of the endoscopic image observation support device.

As illustrated in FIG. 4, the endoscopic image observation support device 100 has functions of an image acquiring unit 111, an image recognition processing unit 112, a degree-of-priority setting unit 113, a display control unit 114, and the like. The functions of the units are implemented by the processor 101 executing a predetermined program (endoscopic image observation support program).

The image acquiring unit 111 performs processing of acquiring, in a time series order, images captured in time series by the endoscope 10. In this embodiment, the images are acquired in real time. That is, the images captured by the endoscope 10 are acquired in real time via the processor device 30.

The image recognition processing unit 112 performs various kinds of recognition processing on the images acquired by the image acquiring unit 111 and generates information to be used for observation support.

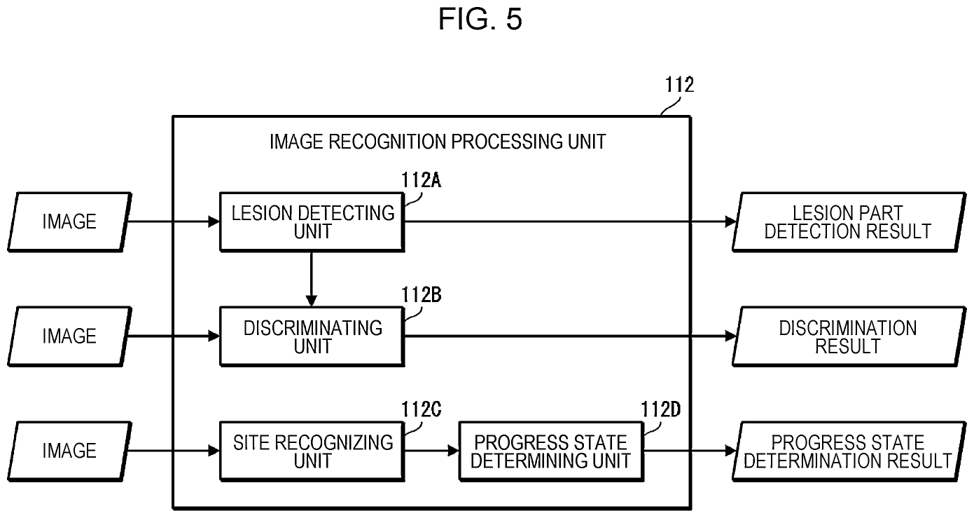

FIG. 5 is a block diagram of main functions of the image recognition processing unit.

As illustrated in FIG. 5, the image recognition processing unit 112 according to this embodiment has functions of a lesion detecting unit 112A, a discriminating unit 112B, a site recognizing unit 112C, a progress state determining unit 112D, and the like.

The lesion detecting unit 112A performs image recognition on an input image to detect a lesion part such as a polyp included in the image. The lesion part includes, in addition to a part that is definitely a lesion part, a part that may be a lesion (e.g., benign tumor or dysplasia), a part having a feature that may be directly or indirectly related to a lesion (e.g., redness), and the like. The lesion detecting unit 112A is constituted by AI, and in particular, is constituted by a trained model that is trained to recognize a lesion part in an image. The detection of the lesion part using the trained model itself is a known technique, and thus, detailed description thereof is omitted. As an example, in this embodiment, the lesion detecting unit 112A is constituted by a trained model using a convolutional neural network (CNN).

The discriminating unit 112B performs discriminating processing on the lesion part detected by the lesion detecting unit 112A. As an example, in this embodiment, processing of estimating the possibility that the lesion part such as a polyp detected by the lesion detecting unit 112A is neoplastic (NEOPLASTIC) or non-neoplastic (HYPERPLASTIC) is performed. The discriminating unit 112B is constituted by AI, and in particular, is constituted by a trained model that is trained to discriminate a lesion part in an image. As an example, in this embodiment, the discriminating unit 112B is constituted by a trained model using a CNN.

The site recognizing unit 112C performs image recognition on the input image to perform processing of recognizing a site included in the image. The site recognizing unit 112C recognizes a site under observation. The site recognizing unit 112C is constituted by AI, and is particularly constituted by a trained model that is trained to recognize a site in an image. As an example, in this embodiment, the site recognizing unit 112C is constituted by a trained model using a CNN.

The progress state determining unit 112D performs processing of determining the observation progress state, based on a site recognition result obtained by the site recognizing unit 112C. Specifically, processing of determining an observation state (observed or unobserved) of a predetermined observation target site is performed. The observation target site is determined for each organ that is an observation target in accordance with an observation (examination) purpose or the like. For example, when the observation target is a stomach, as examples, set observation target sites are (1) esophagogastric junction, (2) lesser curvature immediately below cardia (imaged by J-turn operation), (3) greater curvature immediately below cardia (imaged by U-turn operation), (4) lesser curvature posterior wall from angulus or lower body part (imaged by J-turn operation), (5) pyloric ring from prepyloric region, and (6) greater curvature in lower body part from above. These sites have to be intentionally recorded. In addition, intentional endoscope operations are required at these sites in observation of the stomach. Note that (4) “lesser curvature posterior wall from angulus or lower body part” may also be “lesser curvature in lower body part (imaged by J-turn operation)” in consideration of the fact that it is sometimes not possible to image the gastric angle and the fact that it is not possible to reliably image the posterior wall. In addition, (5) “pyloric ring from prepyloric region” may also be “entire view of antrum” in which importance is attached to whether the antrum is imaged in a bird's eye view rather than imaging the pyloric ring in a pinpoint manner. In addition, (6) “greater curvature in lower body part from above” is not limited to the lower body part, and may be “greater curvature from above” in which importance is attached to the fact that the greater curvature with the folds open is imaged.

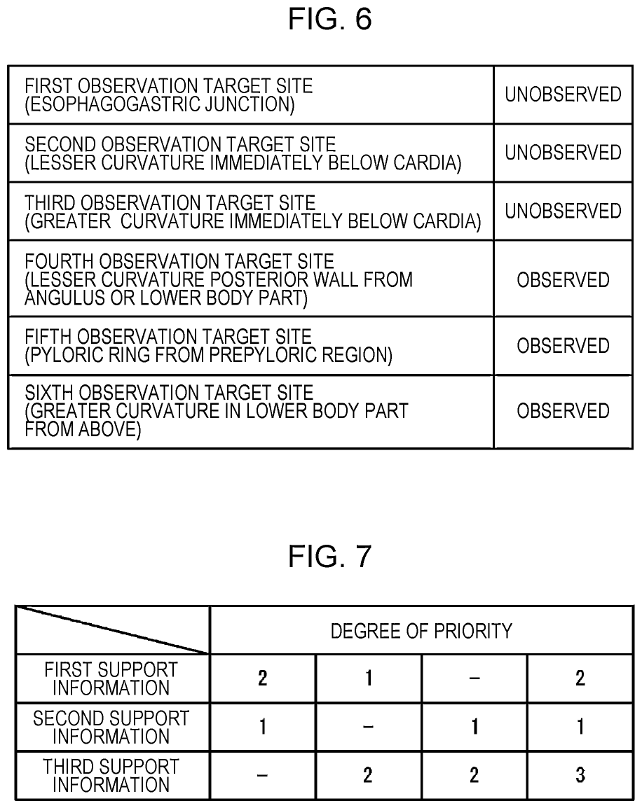

FIG. 6 is a diagram illustrating an example of observation progress state determination results.

As illustrated in FIG. 6, as the observation progress state determination results, whether the respective observation target sites are “observed” or “unobserved” is indicated. An observation target site recognized at least once is regarded as “observed”. On the other hand, an observation target site that is yet to be recognized is regarded as “unobserved”.

The degree-of-priority setting unit 113 performs processing of setting degrees of priority of various kinds of support information to be displayed on the display device 50. The degree of priority is synonymous with a display priority order. The degree of priority is ranked as 1, 2, and 3 in descending order.

As described above, the endoscopic image observation support device 100 according to this embodiment has, as the observation support functions, the function of supporting detection of a lesion part, the function of supporting discrimination, and the function of reporting an observation progress state. With the function of supporting detection of a lesion part, information indicating the position of a lesion part is provided to the user as support information. With the function of supporting discrimination, information indicating a discrimination result is provided to the user as support information. With the function of reporting an observation progress state, information indicating an observation progress state is provided to the user as support information.

The information indicating the position of a lesion part is first support information, the information indicating a discrimination result is second support information, and the information indicating an observation progress state is third support information. When a plurality of pieces of support information are to be displayed on a screen at the same time, the degree-of-priority setting unit 113 sets degrees of priority of the respective pieces of support information. The degree-of-priority setting unit 113 sets the degrees of priority of the respective pieces of support information, based on outputs of the lesion detecting unit 112A, the discriminating unit 112B, and the progress state determining unit 112D. More specifically, the degrees of priority of the respective pieces of support information are set with reference to a table.

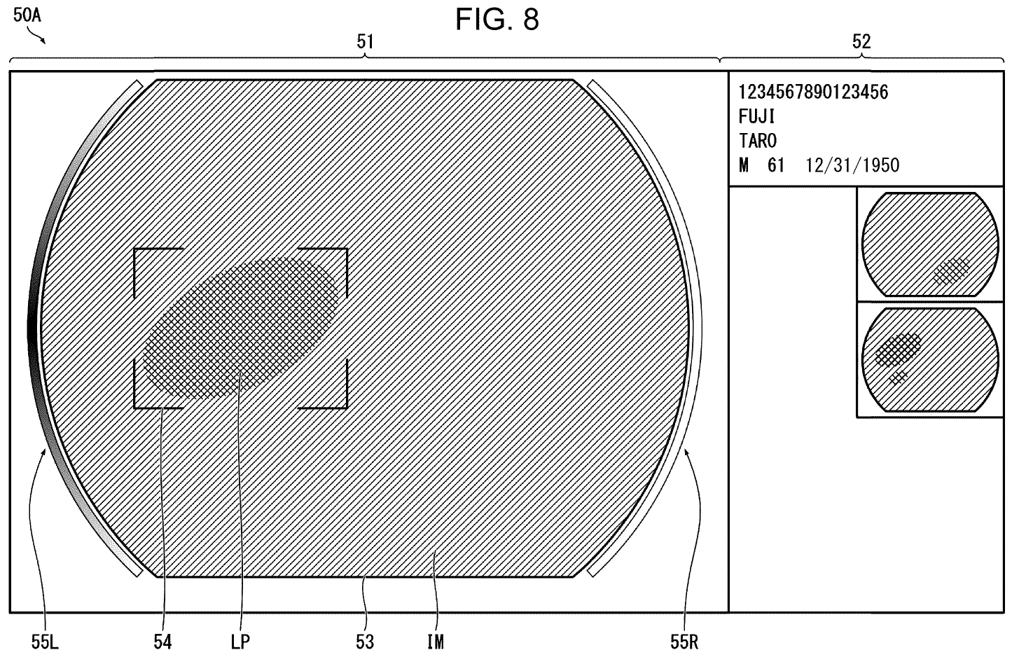

FIG. 7 is a diagram illustrating an example of the table.

In the table, the degrees of priority to be set in accordance with the outputs of the lesion detecting unit 112A, the discriminating unit 112B, and the progress state determining unit 112D are defined.

According to the example illustrated in FIG. 7, in a case where the first support information and the second support information are to be displayed at the same time, the degree of priority of the first support information is set to [2], and the degree of priority of the second support information is set to [1]. In addition, in a case where the first support information and the third support information are to be displayed at the same time, the degree of priority of the first support information is set to [1], and the degree of priority of the third support information is set to [2]. In addition, in a case where the second support information and the third support information are to be displayed at the same time, the degree of priority of the second support information is set to [1], and the degree of priority of the third support information is set to [2]. In addition, in a case where the first support information, the second support information, and the third support information are to be displayed at the same time, the degree of priority of the first support information is set to [2], the degree of priority of the second support information is set to [1], and the degree of priority of the third support information is set to [3].

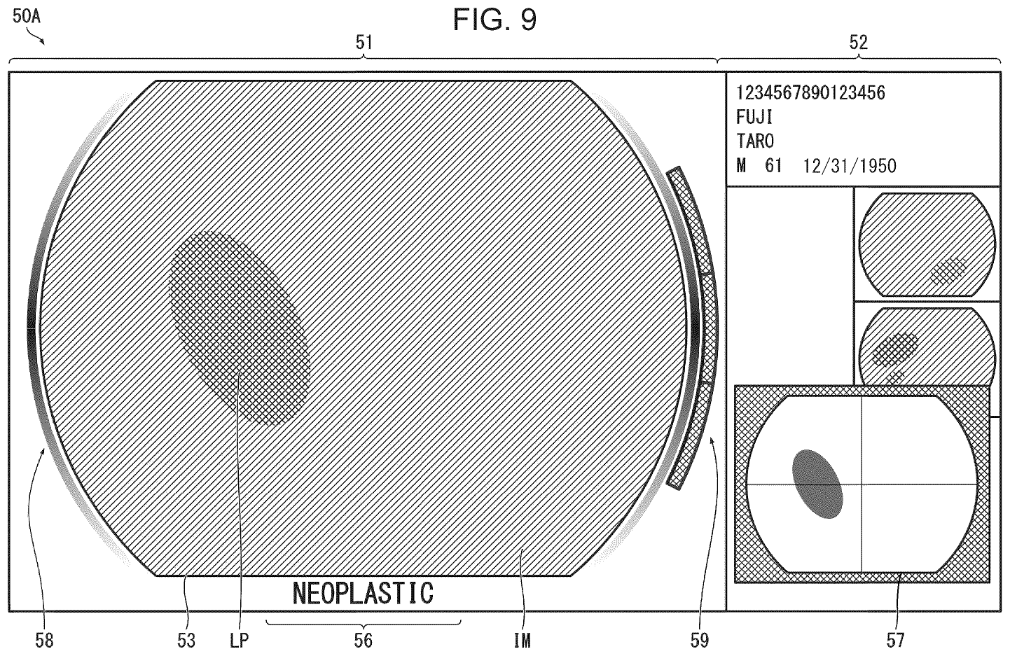

FIG. 8 is a diagram illustrating an example of screen display. FIG. 8 illustrates an example of a case where the display device 50 has a so-called wide screen (horizontally long screen).

As illustrated in FIG. 8, a screen 50A of the display device 50 has a main display region 51 and a sub-display region 52.

The main display region 51 is a region in which an observation image, that is, a live view of an image IM captured by the endoscope, is displayed. The image IM captured by the endoscope is displayed in an observation image display region 53 set in the main display region 51. In this embodiment, the observation image display region 53 has a shape obtained by cutting off the top and bottom of a circle.

The sub-display region 52 is a region used for displaying various kinds of information. For example, information on a subject, a still image captured during observation, or the like is displayed in the sub-display region 52.

FIG. 8 also illustrates an example of display of the first support information. As illustrated in FIG. 8, in this embodiment, a detection box 54 and detection assist circles 55R and 55L are displayed as the first support information.

The detection box 54 is constituted by a rectangular frame and is displayed so as to surround a detected lesion part LP.

The detection assist circles 55R and 55L are constituted by arc-shaped curves displayed along the left and right edges of the observation image display region 53, and a circle closer to the detected lesion part LP is illuminated in a predetermined color (e.g., green). In the example illustrated in FIG. 8, since the lesion part LP is present at a position on the left side of the screen, the detection assist circle 55L on the left side of the screen is illuminated.

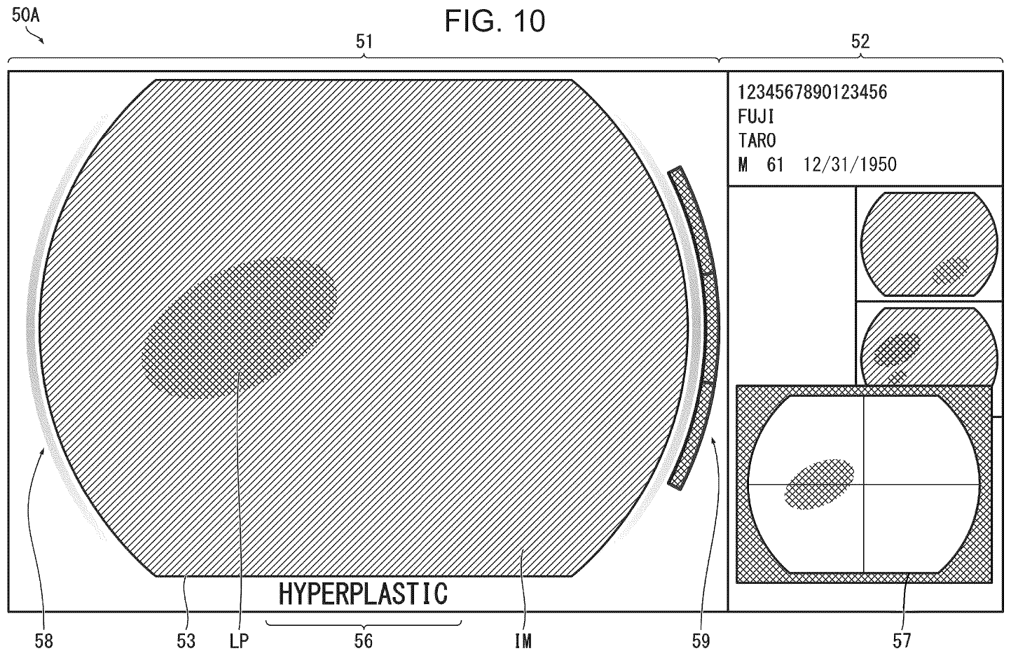

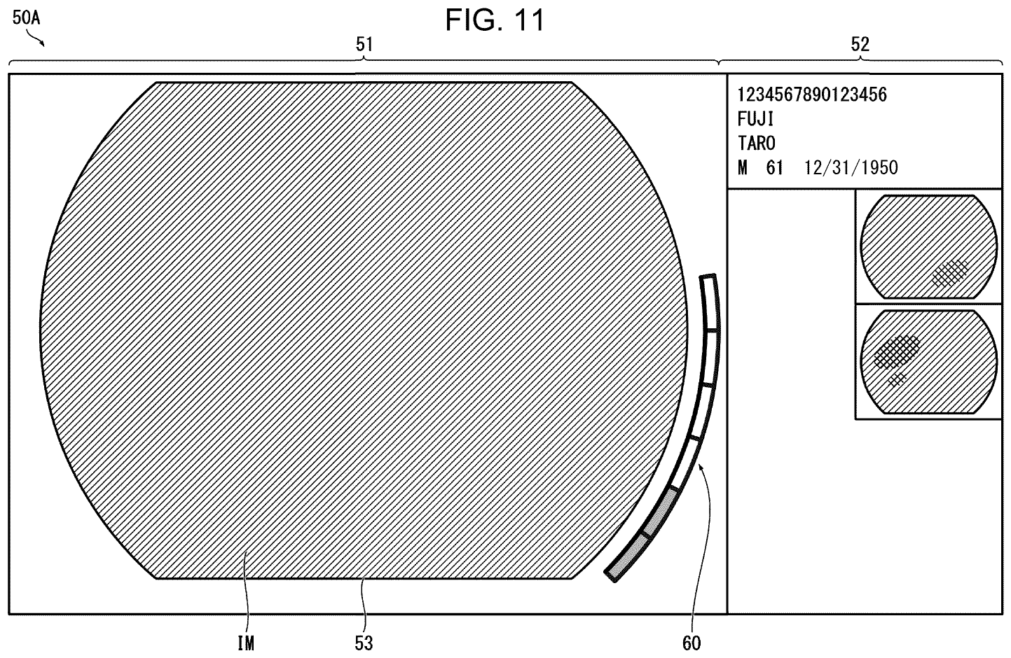

FIGS. 9 and 10 are diagrams illustrating examples of display of the second support information. FIG. 9 illustrates an example of a case where the discrimination result is “neoplastic”, and FIG. 10 illustrates an example of a case where the discrimination result is “non-neoplastic”.

As illustrated in FIGS. 9 and 10, in this embodiment, a discrimination result 56, a position map 57, a discrimination assist circle 58, and a status bar 59 are displayed as the second support information.

As the discrimination result 56, “neoplastic (NEOPLASTIC)” or “non-neoplastic (HYPERPLASTIC)” estimated by the recognition processing is displayed at a predetermined discrimination result display position. In this embodiment, the discrimination result 56 is displayed at a position immediately below the observation image display region 53. If the discrimination result is “neoplastic”, as illustrated in FIG. 9, “NEOPLASTIC” is displayed at the discrimination result display position. On the other hand, if the discrimination result is “non-neoplastic”, as illustrated in FIG. 10, “HYPERPLASTIC” is displayed at the discrimination result display position.

The position map 57 indicates a discrimination target region within an image. In the position map 57, a frame similar to the observation image display region 53 is displayed within a rectangular box, and the discrimination target region is indicated by a predetermined color within the frame. Since the discrimination target region is the region of the lesion part, the region of the lesion part is indicated by the predetermined color. The color is displayed in accordance with the discrimination result. For example, if the discrimination result is “neoplastic”, the discrimination target region is indicated in yellow. On the other hand, if the discrimination result is “non-neoplastic”, the discrimination target region is indicated in green. The position map 57 is displayed at a fixed position. In this embodiment, the position map 57 is displayed at the position map display position set in the sub-display region 52.

The discrimination assist circle 58 is constituted by an arc-shaped curve displayed along the left and right edges of the observation image display region 53 and is illuminated in a color in accordance with the discrimination result. For example, if the discrimination result is “neoplastic”, the discrimination assist circle 58 is illuminated in yellow. On the other hand, if the discrimination result is “non-neoplastic”, the discrimination assist circle 58 is illuminated in green.

The status bar 59 indicates an analysis state, which is neoplastic or non-neoplastic, of the discrimination target region discriminated by the discriminating unit 112B. The status bar 59 is constituted by three arc-shaped blocks arranged at certain intervals along the right edge of the observation image display region 53, and each block is illuminated in accordance with the analysis state. In this embodiment, the analysis state is indicated by three levels (levels 1 to 3). Level 1 is a case of responding to lesions of different types that are mixed. Level 2 is a case of responding to separate lesions of different types. Level 3 is a case of responding to lesions of the same type. In a case of level 1, only the lower one of the three blocks is illuminated. In a case of level 2, two blocks, which are the center one and the lower one, among the three blocks are illuminated. In a case of level 3, all the three blocks are illuminated. FIGS. 9 and 10 illustrate a case where the analysis state is level 3. Note that the discrimination assist circle 58 is illuminated only when the analysis state is level 3.

FIG. 11 is a diagram illustrating an example of display of the third support information.

As illustrated in FIG. 11, in this embodiment, a progress bar 60 is displayed as the third support information. In this embodiment, the progress bar 60 has an arc shape and is arranged along the right edge of the observation image display region 53. The color of the progress bar 60 changes from bottom to top in accordance with the observation progress state.

As described above, in this embodiment, six sites are set as the observation target sites. Each time one observation target site is observed, one gradation of the progress bar 60 changes. That is, the color of one gradation changes. Thus, in this embodiment, six gradations are set in the progress bar 60. FIG. 11 illustrates a state in which observation of two of the six observation target sites is completed. In this case, the color of two gradations changes. When observation of all the observation target sites is completed, the color of all the gradations changes.

When displaying a plurality of pieces of support information at the same time, the display control unit 114 displays the respective pieces of support information, based on the degrees of priority set by the degree-of-priority setting unit 113. In this embodiment, support information with a degree of priority lower than a threshold value is hidden. The threshold value is, for example, 1. Thus, only support information with a degree of priority of 1 is displayed.

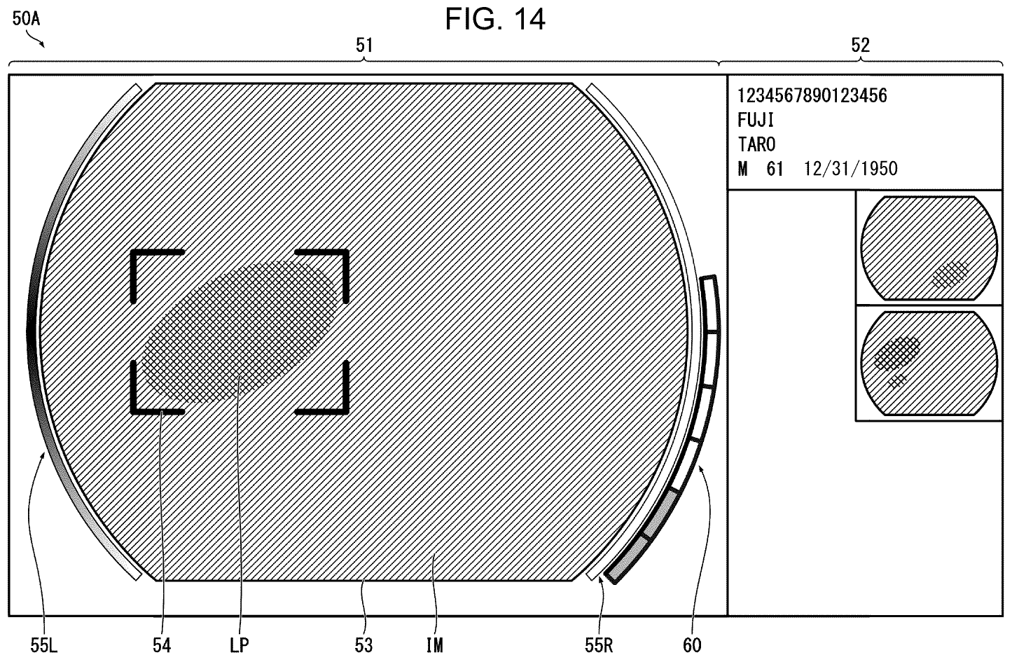

Thus, for example, if a lesion part is detected during display of the third support information (progress bar 60) and the first support information (detection box 54 and detection assist circles 55L and 55R) is to be displayed, the third support information is hidden, and only the first support information is displayed on the screen (display illustrated in FIG. 11 is switched to display illustrated in FIG. 8). Furthermore, if discrimination processing is performed and the second support information (discrimination result 56, position map 57, discrimination assist circle 58, and status bar 59) is to be displayed, the first support information and the third support information are hidden, and only the second support information is displayed on the screen (display illustrated in FIG. 13 is switched to display illustrated in FIG. 9 or FIG. 10).

In this manner, by displaying only one piece of support information, it is possible to prevent the screen from becoming complicated and to provide a user interface with high visibility.

### Operation of Endoscope System

Here, display control of support information will be mainly described.

When observation is started, an image (observation image) captured by the endoscope 10 is displayed on the display device 50 in real time. The observation image is displayed in the observation image display region 53.

If an observation support function is ON, support information is displayed on the screen. ON or OFF of individual support function can be switched. If a plurality of support functions are ON and a plurality of pieces of support information are to be displayed at the same time, the degrees of priority of the respective pieces of support information are set, and display in accordance with the degrees of priority is performed.

The display control of the support information in a case where all the support functions are ON will be described below. That is, the display control of the support information (first support information to third support information) in a case where all of the function of supporting detection of a lesion part, the function of supporting discrimination, and the function of reporting an observation progress state are ON will be described.

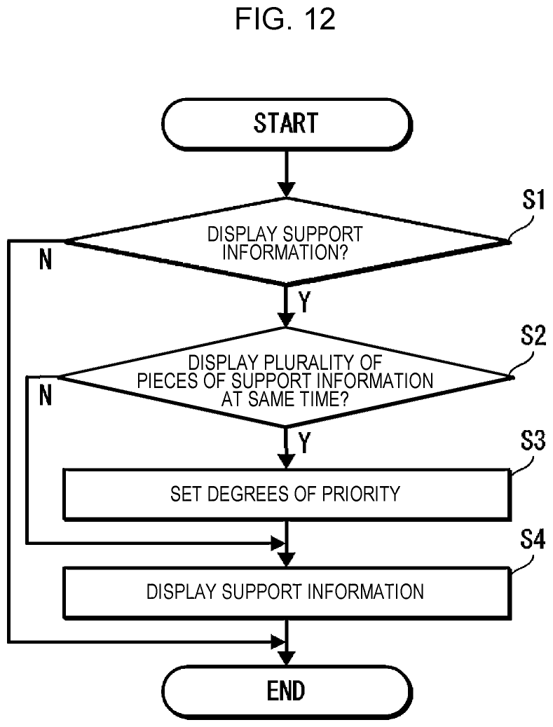

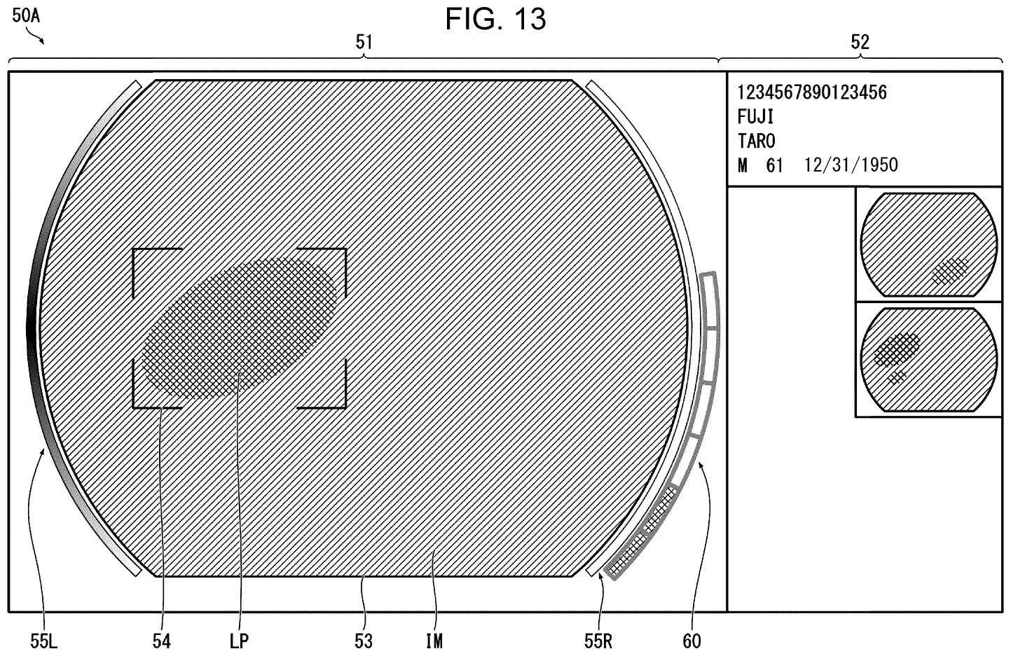

FIG. 12 is a flowchart illustrating a procedure of a support information displaying process.

First, it is determined whether to display support information (step S1). If the support information is to be displayed, it is determined whether a plurality of pieces of support information are to be displayed at the same time (step S2).

If a plurality of pieces of support information are not to be displayed at the same time, that is, if only one piece of support information is to be displayed, the target support information is displayed as it is (step S4).

On the other hand, if a plurality of pieces of support information are to be displayed at the same time, degrees of priority are set for the pieces of support information to be displayed (step S3). The degrees of priority are set with reference to the table (see FIG. 7). The support information is displayed based on the set degrees of priority (step S4). In this embodiment, support information with a degree of priority lower than 1 (threshold value) is hidden. In other words, only support information with a degree of priority of 1 is displayed.

Here, in a case of this embodiment, the first support information (see FIG. 8) is displayed if a lesion part is detected in an image. Similarly, the second support information (see FIG. 9 and FIG. 10) is displayed if discrimination is performed. On the other hand, display of the third support information (see FIG. 11) is started from the start of observation. Thus, when the first support information or the second support information is to be displayed, a plurality of pieces of support information are always displayed. Thus, when the first support information or the second support information is to be displayed, the degrees of priority are always set, and display in accordance with the setting is performed. Specifically, only the first support information or the second support information is displayed. In addition, when all pieces of support information are to be displayed, only the second support information is displayed.

In this manner, according to the endoscope system 1 according to this embodiment, when a plurality of pieces of support information are to be displayed at the same time, the degrees of priority for display are set, and the respective pieces of support information are displayed in accordance with the set degrees of priority. Thus, it is possible to prevent the screen from becoming complicated. In addition, it is possible to provide a user interface with high visibility.

### Modifications

### Display of Support Information in Accordance With Degree of Priority

The above embodiment has described, as an example, a case where display of support information with a degree of priority lower than a threshold value is hidden as display in accordance with degrees of priority. However, the manner of display in accordance with the degrees of priority is not limited to this. For example, a degree of emphasis of display can be changed in accordance with the degrees of priority. For example, the degree of emphasis of display can be changed by changing the display position (including layout changing case), changing the size, changing the luminance, or changing the thickness of a frame or the like. In addition, by combining these as appropriate, display in accordance with the degrees of priority can be performed. For example, when the first support information and the third support information are to be displayed at the same time, the third support information may be displayed with reduced luminance, whereas, when the second support information and the third support information are to be displayed at the same time, only the second support information may be displayed.

FIG. 13 is a diagram illustrating an example of a case where the luminance is changed in accordance with the degrees of priority for display. FIG. 13 illustrates an example of a case where the first support information and the third support information are displayed at the same time. In addition, FIG. 13 illustrates an example of a case of reducing the luminance of support information with a degree of priority lower than a threshold value (threshold value=1). As illustrated in FIG. 13, in this case, the luminance of the third support information (progress bar 60) is reduced. By reducing the luminance of support information with a low degree of priority in this manner, it is possible to prevent the screen from becoming complicated even if a plurality of pieces of support information are displayed at the same time. Note that this example adopts a configuration in which the luminance of support information with a degree of priority lower than the threshold value is reduced. However, it is also possible to adopt a configuration in which the luminance of support information with a degree of priority higher than the threshold value is increased. That is, the luminance of support information with a degree of priority higher than the threshold value may be increased to emphasize the support information. Thus, the luminance of support information with a low degree of priority is relatively reduced, and substantially the same effect is obtained.

For emphasis, in addition to an increase in the luminance, blinking, an increase in size, a change in the display position (display at more noticeable position), or an increase in the thickness of a frame can be performed. FIG. 14 is a diagram illustrating an example of a case where the first support information is displayed in an emphasized manner. FIG. 14 illustrates an example of a case where the detection box 54 is displayed in an emphasized manner by increasing the thickness of the frame constituting the detection box 54. In addition, the degree of emphasis of the first support information can also be changed by changing the length of the frame constituting the detection box 54 (length of line extending from each corner along each side).

In addition, if a plurality of pieces of support information based on one support function are to be displayed, types of support information to be displayed may be changed in accordance with degrees of priority. For example, in the above embodiment, in the function of supporting detection of a lesion part, the detection box 54 and the detection assist circles 55R and 55L are displayed as the first support information. In this case, in accordance with the degrees of priority, only either the detection box 54 or the detection assist circles 55R and 55L can be displayed, both the detection box 54 and the detection assist circles 55R and 55L can be displayed, or both the detection box 54 and the detection assist circles 55R and 55L can be hidden.

### Modifications of Third Support Information

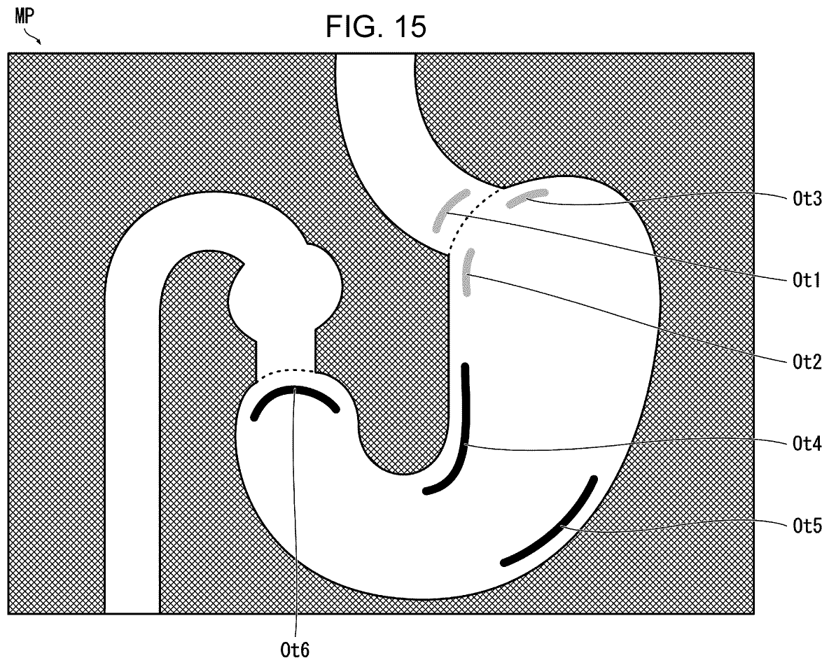

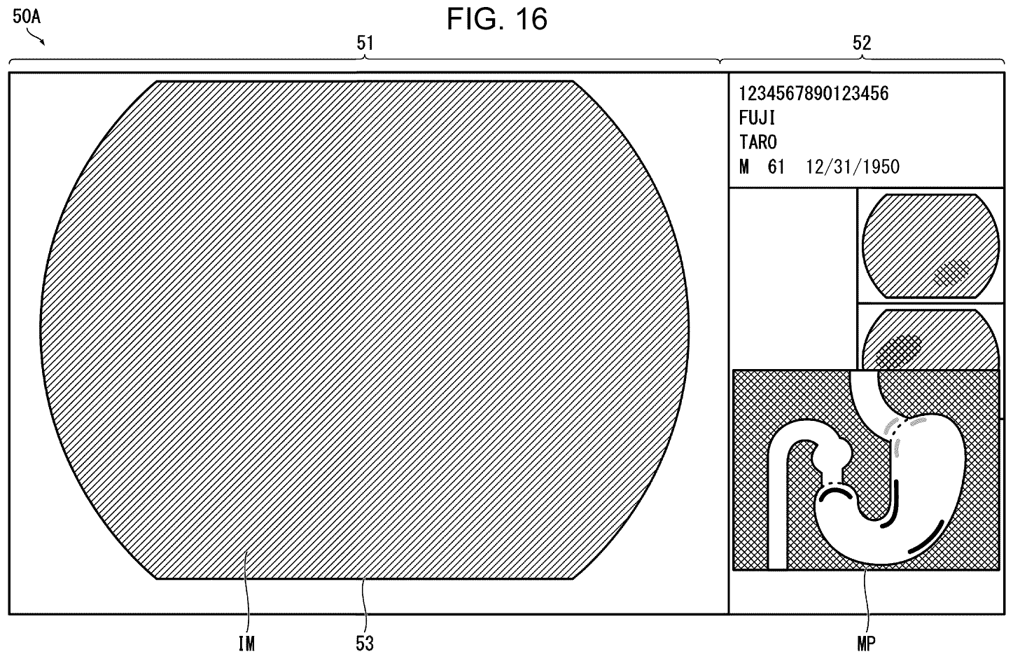

FIG. 15 is a diagram illustrating another example of the third support information.

FIG. 15 illustrates an example of a case of indicating an observation progress state by using an observation state display map MP. The observation state display map MP is generated using a schema diagram of an observation target organ. FIG. 15 illustrates an example of a case where the observation target is a stomach. Specifically, the schema diagram of the observation target organ (stomach in this example) is displayed in a rectangular box, and observation target sites Ot1 to Ot6 are indicated by lines on the schema diagram. FIG. 15 illustrates an example of a case where the first observation target site Ot1 is “esophagogastric junction”, the second observation target site Ot2 is “lesser curvature immediately below cardia”, the third observation target site Ot3 is “greater curvature immediately below cardia”, the fourth observation target site Ot4 is “lesser curvature posterior wall from angulus or lower body part”, the fifth observation target site Ot5 is “pyloric ring from prepyloric region”, and the sixth observation target site Ot6 is “greater curvature in lower body part from above”. The lines indicating the observation target sites Ot1 to Ot6 are displayed in different colors depending on whether the observation target sites are “observed” or “unobserved”. For example, the line of the “unobserved” observation target site is displayed in gray, and the line of the “observed” observation target site is displayed in green (displayed in black in FIG. 15). FIG. 15 illustrates an example of a case where the first observation target site Ot1, the second observation target site Ot2, and the third observation target site Ot3 are “unobserved”, and the fourth observation target site Ot4, the fifth observation target site Ot5, and the sixth observation target site Ot6 are “observed”. In this manner, by displaying the lines indicating the observation target sites on the observation state display map MP and displaying “observed” and “unobserved” in a distinguishable manner by the colors of the lines, it is possible to grasp at a glance the observation state of each observation target site and the overall progress state.

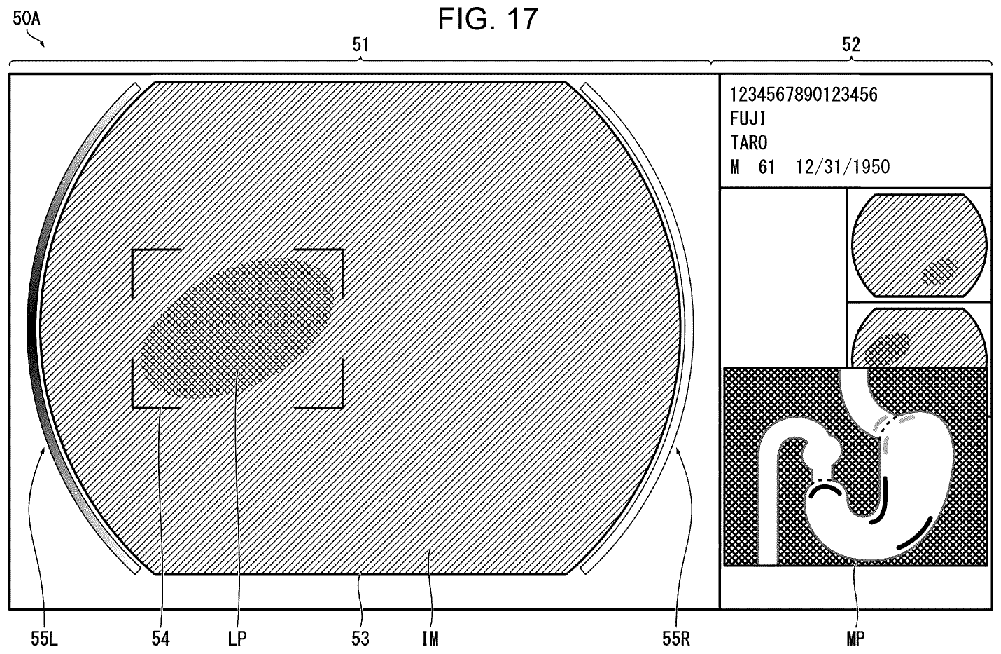

FIG. 16 is a diagram illustrating an example of display of the observation state display map on the screen. FIG. 16 illustrates an example of a case where the observation state display map MP is normally displayed (displayed without degree of priority).

The observation state display map MP is displayed at a predetermined position and in a predetermined size. FIG. 16 illustrates an example of a case where the observation state display map MP is displayed in the sub-display region 52.

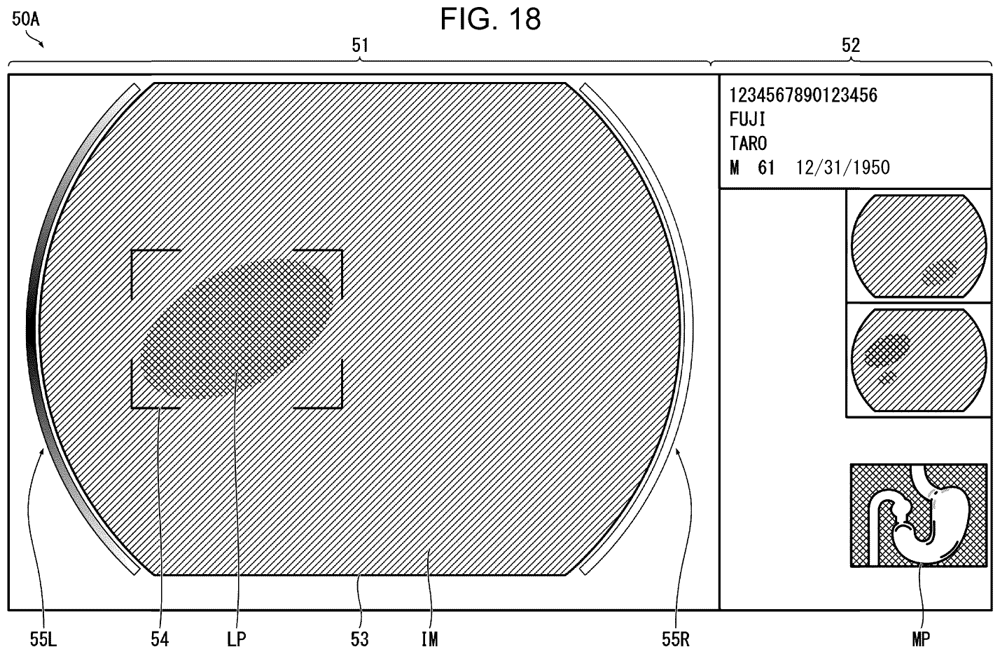

FIG. 17 is a diagram illustrating an example of display of support information in accordance with degrees of priority. FIG. 17 illustrates an example of a case where the first support information and the third support information are displayed at the same time. In addition, FIG. 17 illustrates an example of a case where, if the degree of priority of the first support information is 1 and the degree of priority of the third support information is 2, the luminance of support information with a degree of priority lower than a threshold value (threshold value=1) is reduced. As illustrated in FIG. 17, in this case, the luminance of the observation state display map MP, which is the third support information, is reduced.

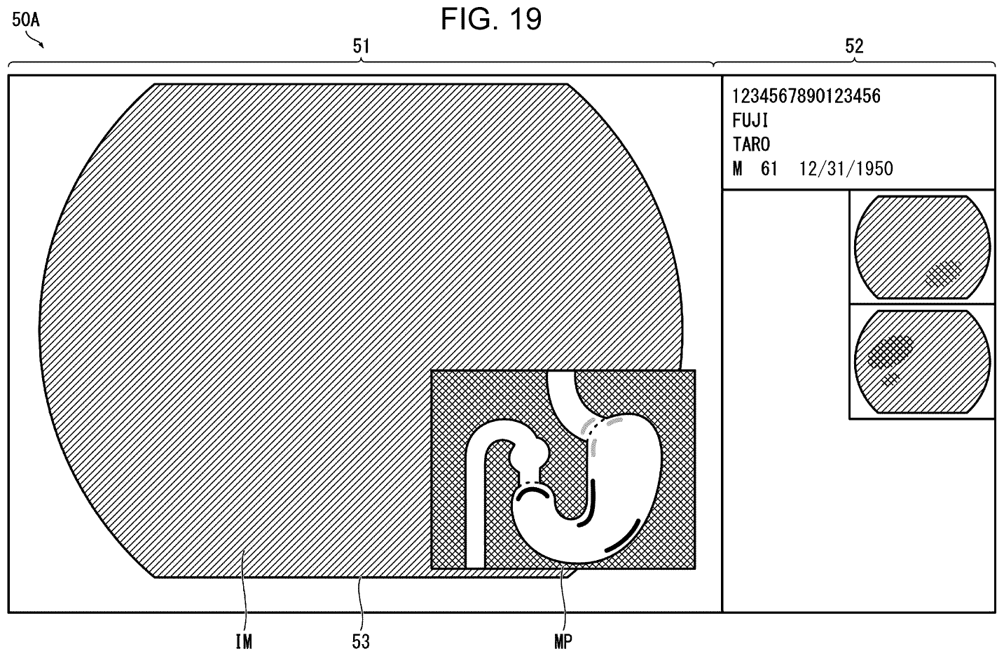

FIG. 18 is a diagram illustrating another example of display of support information in accordance with degrees of priority. FIG. 18 illustrates an example of a case where the first support information and the third support information are displayed at the same time. In addition, FIG. 18 illustrates an example of a case where, if the degree of priority of the first support information is 1 and the degree of priority of the third support information is 2, the position and size of support information with a degree of priority lower than a threshold value (threshold value=1) are changed. That is, an example of a case of display with the size reduced and away from the observation image display region 53 is illustrated. As illustrated in FIG. 18, in this case, the observation state display map MP, which is the third support information, is displayed to be smaller than in a normal case (see FIG. 16) and is displayed at a position more away from the observation image display region 53 than in a normal case (see FIG. 16).

FIG. 19 is a diagram illustrating an example of a case where the third support information is displayed in an emphasized manner.

FIG. 19 illustrates an example of a case of changing the position of the observation state display map MP, which is the third support information. In a case where the position is changed, as illustrated in FIG. 19, the observation state display map MP is displayed to be closer to the observation image display region 53 than in a normal case (see FIG. 16). In the example illustrated in FIG. 19, the observation state display map MP is moved horizontally to be close to the observation image display region 53.

Note that the degrees of priority are preferably changed when display content of the third support information is updated. For example, regarding the observation state display map MP, the degrees of priority are preferably changed when one of the observation target sites is changed from the “unobserved” state to the “observed” state. In addition, the degrees of priority are preferably changed when one gradation of the progress bar 60 is increased. In this case, the degree of priority is changed to be higher. For example, in a case where the display content of the third support information is updated in a state where the first support information and the third support information are displayed, the degree of priority of the first support information is decreased from 1 to 2, whereas the degree of priority of the third support information is increased from 2 to 1. Thus, a user can more easily recognize that the third support information is updated.

Note that in a case where the degrees of priority are changed based on updating of the display content of the third support information in this manner, it is preferable to return to the setting of the original degrees of priority after a predetermined time elapses from the change. For example, in a case of the above example, after a predetermined time elapses from the change, the degree of priority of the first support information is returned to 1, and the degree of priority of the third support information is returned to 2. Thus, the degrees of priority can be switched appropriately.

As a display manner in accordance with the degree of priority of the third support information, display using the progress bar 60 and the observation state display map MP can be switched for display. For example, it is possible to adopt a configuration in which the observation state display map MP is used in a normal display manner, and the progress bar 60 is displayed when the degree of priority is reduced. Alternatively, it is possible to adopt a configuration in which the progress bar 60 is used in the normal display manner, and the observation state display map MP is displayed when the degree of priority is increased.

In addition, in the above embodiment, the third support information is displayed by using the progress bar in a form along the edge of the observation image display region 53. However, the shape and display position of the progress bar are not limited to these. For example, a linear progress bar can also be used for display. In addition, a circular progress bar can also be used for display.

Instead of the progress bar or the like, or in addition to the progress bar or the like, the third support information may be displayed by indicating an observation progress state by a numerical value (e.g., percentage).

### Second Embodiment

Degrees of priority of display of respective pieces of support information are set based on importance, usefulness, or the like of the information. However, the importance, usefulness, or the like of the respective pieces of support information changes depending on the situation. Thus, the degrees of priority can be set more appropriately in consideration of the situation. This embodiment will describe a case where the degrees of priority are set (including change) in accordance with the situation.

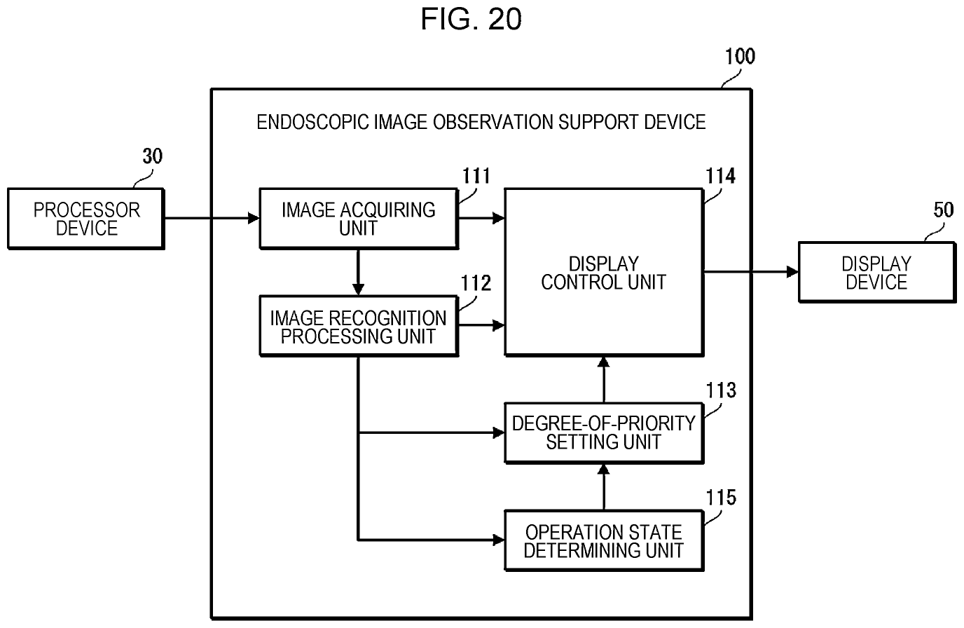

FIG. 20 is a block diagram of main functions of the endoscopic image observation support device.

As illustrated in FIG. 20, the endoscopic image observation support device 100 according to this embodiment further has a function of an operation state determining unit 115.

The operation state determining unit 115 determines an operation state of the endoscope 10. In this embodiment, the operation state of the endoscope 10 is determined based on a site recognition result obtained by the site recognizing unit 112C and a progress state determination result obtained by the progress state determining unit 112D. Specifically, it is determined whether an observed site is being observed. Whether an observed site is being observed is determined based on an observation progress state determination result and a site recognition result.

The degree-of-priority setting unit 113 sets or changes the degrees of priority of the respective pieces of support information, based on outputs of the lesion detecting unit 112A, the discriminating unit 112B, and the progress state determining unit 112D and a determination result obtained by the operation state determining unit 115. For example, if an observed site is being observed, the degree of priority of the first support information is reduced. As a result, for example, when the first support information and the third support information are to be displayed, the set degree of priority changes depending on whether an observed site is being observed. That is, if an observed site is not being observed, as in a normal case, the degree of priority of the first support information is set to 1, and the degree of priority of the third support information is set to 2. On the other hand, if an observed site is being observed, the degree of priority of the first support information is set to 2, and the degree of priority of the third support information is set to 1.

In this manner, by setting or changing the degrees of priority of display of the respective pieces of support information in accordance with the situation, useful information can be displayed appropriately. Thus, even if a plurality of pieces of support information are displayed, it is possible to prevent the screen from becoming complicated and to provide a user interface with high visibility.

Note that the above embodiment has described, as an example, a case where the degrees of priority are set or changed depending on whether an observed site is being observed. However, the determination of the situation is not limited to this. In addition, for example, it is possible to determine whether a lesion part is being observed, whether a region in which the lesion part is detectable is being observed, whether there is an unobserved site, whether a treatment is being performed, or the like, and to set or change the degrees of priority in accordance with the determination result. In addition, these situations are preferably determined based on an image captured by the endoscope. For example, whether a lesion part is being observed can be determined by using a lesion part recognition result. In addition, whether a region in which the lesion part is detectable is being observed can be determined by using the site recognition result. In addition, whether there is an unobserved site can be determined by using the observation progress state determination result. In addition, whether a treatment is being performed can be determined by, for example, detecting the presence or absence of a treatment tool such as forceps or a snare in the image. In this case, if a treatment tool is detected in the image, it is determined that the treatment is being performed.

In addition, the operation state of the endoscope can be determined by using operation information of the operating unit of the endoscope 10, information manually input by a user, or the like.

### Other Embodiments

### Setting of Degrees of Priority

It is possible to adopt a configuration in which degrees of priority are set in comparison with other pieces of support information. In this case, it is preferable to set the degrees of priority of display of the respective pieces of support information in accordance with the following criteria.

When the first support information is to be displayed, the degree of priority of the third support information is reduced, and the luminance thereof is reduced, or the third support information is hidden. Similarly, when the second support information is to be displayed, the degree of priority thereof is reduced, the luminance thereof is reduced, or the third support information is hidden.

In addition, when the display content of the third support information is updated, the degree of priority thereof is increased, and the third support information is displayed in a blinking manner or is displayed at a different display position.

In addition, when an observed site is being observed (imaged) and when a lesion part is being observed, the degree of priority of the first support information is reduced.

In addition, regarding the first support information and the second support information, the degree of priority of the first support information is relatively increased in a normal case (removal or the like), whereas the degree of priority of the second support information is relatively increased in a case of observation.

In addition, the following criteria can be adopted.

The degree of priority of the third support information is preferably increased in the following scene. That is, the scene is a case where there is no lesion part to be detected and a case where a user is not observing. The case where there is no lesion part to be detected includes a case where an observed site is not compatible with the function of supporting detection of a lesion part and a case where a lesion part is undetected. In addition, the case where a user (surgeon) is not observing includes a case where pretreatment is being performed, a case where an insertion operation of the endoscope is being performed, and a case where an image of an already observed site is being captured (observed).

For example, when a lesion part is being observed, the degree of priority of the second support information is preferably increased.

For example, when the observation purpose is a screening examination, the degree of priority of the first support information is preferably increased.

In addition, the above embodiments adopt configurations in which, when a plurality of pieces of support information are to be displayed at the same time, the degrees of priority of display thereof are set. However, the degrees of priority of display of the respective pieces of support information may be set regardless of whether the pieces of support information are to be displayed at the same time. In this case, for example, in accordance with the above criteria, the degrees of priority of the respective pieces of support information are dynamically changed, and the respective pieces of support information are displayed.

In addition, when the degrees of priority are set, the same degree of priority may be set for a plurality of pieces of support information. For example, when degrees of priority are set for three pieces of support information, the same priority can be set for two pieces of support information. In addition, for example, a degree of priority of “high” or “low” can be individually set for each piece of support information.

### Case of Individual Display

When an individual piece of support information is to be displayed, a display manner thereof can be changed upon various events. For example, as described above, the third support information can be displayed in an emphasized manner for a certain period of time at the timing of updating the content thereof. The other pieces of support information can also be displayed in an emphasized manner for a certain period of time from the start of display in the same manner.

In addition, when an individual piece of support information is to be displayed, the display manner thereof can be changed with reference to outputs of other support functions. In this case, the display manner can be changed by using the above criteria.

### Support Functions to Be Provided

The above embodiments have described, as an example, a case where the observation supporting functions include the function of supporting detection of a lesion part, the function of supporting discrimination, and the function of reporting an observation progress state. However, the support functions to be provided are not limited to these. It is only necessary to provide at least two support functions.

### Observation Target

The above embodiments have described a system for observing (examining) an upper digestive organ. However, the application of the present invention is not limited to this. For example, the present invention is similarly applicable to a system for observing a lower digestive tract such as a large intestine.

### Hardware Configuration

The functions of the endoscopic image observation support device can be implemented by various processors. Various processors include a central processing unit (CPU) and/or a graphic processing unit (GPU), which are general-purpose processors functioning as various processing units by executing programs, a programmable logic device (PLD), which is a processor in which the circuit configuration is changeable after manufacture, such as field programmable gate array (FPGA), a dedicated electric circuit, which is a processor having a circuit configuration that is specially designed to execute specific processing, such as an application specific integrated circuit (ASIC), and the like. The program is synonymous with software.

One processing unit may be constituted by one of these various processors, or may be constituted by two or more processors of the same type or different types. For example, one processing unit may be constituted by a plurality of FPGAs or a combination of a CPU and an FPGA. In addition, a plurality of processing units may be constituted by one processor. Firstly, as an example of constituting a plurality of processing units using one processor, there is a form in which one processor is constituted by a combination of one or more CPUs and software, and the processor functions as a plurality of processing units, as typified by a computer used as a client, a server, or the like. Secondly, there is a form of using a processor that implements the functions of the entire system including a plurality of processing units with one integrated circuit (IC) chip, as typified by a system on chip (SoC) or the like. In this manner, various processing units are constituted by one or more of the above various processors in terms of hardware configuration.

### REFERENCE SIGNS LIST

1 endoscope system
10 endoscope
20 light source device
30 processor device
31 endoscope control unit
32 light source control unit
33 image processing unit
34 input control unit
35 output control unit
40 input device
50 display device
50A screen of display device
51 main display region
52 sub-display region
53 observation image display region
54 detection box
55L detection assist circle
55R detection assist circle
56 discrimination result
57 position map
58 discrimination assist circle
59 status bar
60 progress bar
100 endoscopic image observation support device
101 processor
102 main memory
103 auxiliary storage
104 input/output interface
111 image acquiring unit
112 image recognition processing unit
112A lesion detecting unit
112B discriminating unit
112C site recognizing unit
112D progress state determining unit
113 degree of priority setting unit
114 display control unit
115 operation state determining unit
IM image
LP lesion part
MP observation state display map
Ot1 first observation target site
Ot2 second observation target site
Ot3 third observation target site
Ot4 fourth observation target site
Ot5 fifth observation target site
Ot6 sixth observation target site
S1-S4 procedure of support information displaying process

What is claimed is:

1. An endoscopic image observation support device that supports observation of an image captured by an endoscope, the endoscopic image observation support device comprising:
a processor configured to:
cause a display device to display the image;
set degrees of priority of a plurality of pieces of support information to be displayed on the display device; and
cause the display device to display the plurality of pieces of support information based on the degrees of priority.

2. The endoscopic image observation support device according to claim 1, wherein
the plurality of pieces of support information include at least one of information indicating a position of a lesion part, information indicating a discrimination result, or information indicating an observation progress state.

3. The endoscopic image observation support device according to claim 2, wherein
the information indicating an observation progress state is displayed using a progress bar.

4. The endoscopic image observation support device according to claim 2, wherein
the information indicating an observation progress state is displayed using a schema diagram of an observation target organ.

5. The endoscopic image observation support device according to claim 1, wherein
the processor is configured to cause the display device to display the plurality of pieces of support information at positions in accordance with the degrees of priority.

6. The endoscopic image observation support device according to claim 1, wherein
the processor is configured to cause the display device to display the plurality of pieces of support information in sizes in accordance with the degrees of priority.

7. The endoscopic image observation support device according to claim 1, wherein
the processor is configured to cause the display device to display the plurality of pieces of support information at luminances in accordance with the degrees of priority.

8. The endoscopic image observation support device according to claim 1, wherein
the processor is configured to emphasize display of one of the plurality of pieces of support information with a degree of priority higher than a threshold value.

9. The endoscopic image observation support device according to claim 8, wherein
the processor is configured to blink display of the one of the plurality of pieces of support information with the degree of priority higher than the threshold value.

10. The endoscopic image observation support device according to claim 1, wherein
the processor is configured to hide display of one of the plurality of pieces of support information with a degree of priority lower than a threshold value.

11. The endoscopic image observation support device according to claim 1, wherein
the processor is configured to change the degrees of priority in accordance with an operation state of the endoscope.

12. The endoscopic image observation support device according to claim 11, wherein
the processor is configured to determine the operation state of the endoscope based on the image.

13. The endoscopic image observation support device according to claim 11, wherein
the operation state of the endoscope is whether an observed site and/or a lesion part is being observed.

14. The endoscopic image observation support device according to claim 11, wherein
the operation state of the endoscope is whether a region in which a lesion part is detectable is being observed.

15. The endoscopic image observation support device according to claim 11, wherein
the operation state of the endoscope is whether there is an unobserved site.

16. The endoscopic image observation support device according to claim 11, wherein
the operation state of the endoscope is whether treatment is being performed.

17. The endoscopic image observation support device according to claim 1, wherein
the processor is configured to change the degrees of priority when display content of a specific piece of support information is updated.

18. An endoscope system comprising:
an endoscope;
a display device; and
the endoscopic image observation support device according to claim 1.
